(19) United States
(12) Patent Application Publication
(10) Pub. No.: US 2018/0230552 A1
(43) Pub. Date: Aug. 16, 2018

(54) Method for Detecting Nucleic Acids in Samples Containing Biological Material

(71) Applicant: Glycotope GmbH, Berlin (DE)

(72) Inventors: Steffen Goletz, Berlin (DE); Doreen Weigelt, Berlin (DE); Jana Langthaler, Mannheim (DE); Helmut Troester, Mannheim (DE)

(21) Appl. No.: 15/513,475

(22) PCT Filed: Sep. 22, 2015
(86) PCT No.: PCT/EP2015/071662

§ 371 Date: Mar. 22, 2017

(30) Foreign Application Priority Data
Sep. 22, 2014 (LU) 92552

(51) Int. Cl. C12Q 1/68; C12N 15/10

(52) U.S. Cl. CPC C12Q 1/6888; C12N 15/1017; C12N 15/101; C12Q 1/6806

(57) ABSTRACT

This invention relates to a detection method for nucleic acids in samples that contain biological material, as well as to a kit having components with which the detection method can be carried out. In samples that contain biotechnologically produced biological material, for example, the detection method is suitable for detecting nucleic acids of host cells that were used for the production of the material.

### CROSS-REFERENCE TO RELATED APPLICATIONS

This application is the U.S. national phase application filed under 35 U.S.C. § 371 claiming benefit to International Patent Application No. PCT/EP2015/071662, filed Sep. 22, 2015, which is entitled to priority to LU 92552, filed Sep. 22, 2014, each of which application is hereby incorporated herein by reference in its entirety.

### BACKGROUND

Biopharmaceutical products are produced by fermentation, either by means of microbial or eukaryotic host cells, in in part complex media. At harvest, preparations of active substances from such fermentations routinely contain many biological molecules from the host cells, which are present as contaminants, in addition to the desired product. These contaminants include lipids, carbohydrates, or components of the bacterial or fungal cell wall or of the eukaryotic cell membrane, as well as host DNA.

As the desired product is being isolated from either host cells or fermentation broth (e.g., supernatant), these contaminants are often isolated and/or cleaned along with the desired product. It is furthermore known that biological molecules originating from some host cells can have toxic effects. Hence in order to prevent possible adverse effects, the removal of host cells and all other contaminating materials is desirable. The complete removal of all contaminants originating from host cells is technologically demanding and in part difficult, but nevertheless mandatory under regulations.

Regulatory authorities for the approval of foods, diagnostics, or medicines therefore define still permissible values of by-products in biotechnologically produced products, including threshold values for host cell DNA in such products. The US Food and Drug Administration (FDA) sets an upper limit of 100 pg of host cell DNA per therapeutic dose (US Food and Drug Administration (1997) Points to consider in the manufacture and testing of monoclonal antibody products for human use), which is relevant in, for example, the administration of therapeutic antibodies at higher doses, since such antibodies are often administered intravenously in larger volumes. The World Health Organization (WHO) has also published guidelines on the upper limit of up to 10 ng host cell DNA/dose (World Health Organization, Technical Reports (1987), Report of a WHO Study group. Acceptability of cell substrates for production of biologicals.).

Manufacturers of biotechnologically generated products (e.g., medicines diagnostics, or foods) are obligated to state whether the residual host cell DNA contained in the product is within the still acceptable limits for a specific product.

Various methods are available for detecting or determining the amount of host cell DNA, such as PicoGreen analysis, hybridization analysis, or quantitative PCR (Mehta and Keer (2007), BioProcess International: 44-58). Although the currently available methods for determining host cell DNA fulfill the legal requirements and the safety requirements, in industry there is a growing need for detailed information on the performance characteristics of these methods. Hence it is particularly desirable to have more sensitive methods available that are capable of quantitatively detecting even the most minute traces of nucleic acids (i.e., in the picogram to the femtogram range) in samples that contain biological material. Such methods are not only of value for detecting host cell DNA in biotechnologically generated products, but also for use in general (e.g., forensics) to detect nucleic acids in a sample that contains biological material.

The object of this invention is therefore that of providing an economical and sensitive method for detecting a nucleic acid in a sample that contains biological material. This object is achieved by the embodiments and subject matter contained in the claims and in the following description, which are illustrated by the examples and figures.

### DETAILED DESCRIPTION

To their own surprise, the inventors of this invention were able to achieve the object thereof such that henceforth a method for detecting a nucleic acid in a sample that contains biological material will be available that advantageously surpasses the previous lower limits. The method and kit of the invention preferably reach ranges of a mere 0.08-2.75 fg/μl sample, which surpasses the detection limit of 10 fg bacterial DNA and 5 pg mammalian DNA of the reputedly very sensitive quantitative PCR (qPCR) method. The method and the kit according to the invention thus advantageously enable compliance with the upper limit set by the regulatory authorities (e.g., the FDA) of 100 pg host cell DNA per therapeutic dose of a biotechnologically generated product, in particular of an antibody, for example, even when the latter is administered in greater dosages. For example, antibodies in particular are administered in larger volumes of, say, 200 ml, meaning that only 0.5 pg host cell DNA may be contained per ml. Because the lower detection limit of standard methods is 0.5 pg host cell DNA, they are unable to detect concentrations below this limit, hence it cannot be shown whether a biotechnologically produced product to be administered to human beings contains host cell DNA in concentrations less than 0.5 pg.

In particular, to their surprise the inventors found that the sequence of steps (a) protease digestion of the sample, (b) concentration of the sample, (c) enrichment of the nucleic acid from the sample, and (d) detection of the nucleic acid by means of the quantitative polymerase chain reaction (qPCR) method surpasses the detection limit of commercial kits (such as the ones used in forensics, for example) for detecting nucleic acids, even though these kits have already been optimized (see Example 4). The inventors furthermore found that steps (a), (b) and (c) are decisive for being able to detect a quantity of nucleic acid in a sample that is less than 0.5 pg/ml. Neither the sequence of the steps of the method according to the invention nor the finding that steps (a), (b) and (c) are decisive for reliably detecting a nucleic acid present in a sample in a quantity of less than 0.5 pg/ml were known or suggested in the prior art.

Accordingly, this invention relates to a method for detecting a nucleic acid in a sample that contains biological material, comprising
(a) Treatment of the sample with protease;
(b) Concentration of the sample;
(c) Enrichment of the nucleic acid from the sample; and
(d) Detection of the nucleic acid by means of quantitative PCR.

The method is generally an in vitro or ex vivo method.

The term “detection” comprises both the detection and the quantification of a nucleic acid in a sample that contains biological material. The method according to the invention does not necessarily have to result in the positive detection of a nucleic acid, because in a sample that contains biological material, there could either be no nucleic acids contained at the outset or else the quantity of nucleic acid contained is so small that it cannot even be detected with the method of the invention. The “detection” with the aid of the method according to the invention therefore also comprises determining whether or not a nucleic acid is contained in a sample that contains biological material.

Accordingly, this invention also relates to a method for determining whether a sample that contains biological material contains a nucleic acid, said method comprising
(a) Treatment of the sample with protease;
(b) Concentration of the sample;
(c) Enrichment of the nucleic acid from the sample; and
(d) Determination, by means of quantitative PCR, whether a nucleic acid is contained in the sample.

Preference is given to a nucleic acid being detected or determined not only qualitatively but also quantitatively with a method according to the invention, as described in more detail herein.

Preferably in addition or alternatively, a method according to the invention is used
to detect a nucleic acid in biological material (preferably a nucleic acid from a host cell in biological material) that will be administered to human beings;
to determine whether biological material for administration to human beings is essentially free of nucleic acids (in particular DNA), preferably free of host cell nucleic acids (in particular DNA); or
to quantify nucleic acids, preferably host cell nucleic acids, in a sample.

The PCR can be monitored in the case of qPCR or real-time PCR. This is done by means of a fluorescence signal that becomes stronger as the number of PCR products (amplicons) formed increases. The fluorescence signal is thus proportional to the content of the PCR product. The increasing content of the product can be visualized as a curve by measuring the fluorescence signal with each PCR cycle. Quantitative and qualitative nucleic acid analyses can be performed using this curve. A qPCR amplification curve can be divided into three zones. In the beginning, the fluorescence signal of the background exceeds that of the actual amplification. In each PCR cycle, the amplicons propagate and the fluorescence signal thus gets stronger. From a certain point on, the fluorescence signal is greater than the background signal. This point is known as the crossing point (Cp). The exponential phase of the qPCR curve also starts at this time. In the exponential phase there are ca. 1000 amplified molecules in a reaction vessel. By determining the time of the Cp, it becomes possible to quantify DNA by means of qPCR. The Cp is preferably expressed as a cycle number. Lastly, the curve ends in the plateau phase. In the plateau phase, fewer and fewer amplicons are formed and the DNA synthesis ultimately stagnates.

The qPCR fluorescence signals can be generated in different ways. The most commonly used systems are intercalating fluorescent dyes that bind to double-stranded DNA on the one hand, and fluorescent-marked oligonucleotides on the other hand, which bind specifically to the DNA and do not emit a measurable fluorescence until they are degraded in the course of the PCR reaction. A qPCR method using SYBR Green and one using hydrolysis probes exemplify each of the two methods, respectively.

A quantification, which is preferably used in conjunction with a method according to the invention, functions as follows: in order to determine the concentration of a sample, the crossing point of the unknown sample is compared to the Cp value of a pre-defined standard. To this end, the standard is initially taken as the standard curve. For this purpose, a dilution series of the standard is prepared and measured by quantitative PCR (qPCR). The software preferably makes it possible to state the concentrations of the standard, on the basis of which the standard curve is automatically calculated. In this process, the log of the concentrations is plotted against the Cp values. If an unknown sample is then measured, the concentration can be determined by comparing the Cp value to the standard curve (see FIG. 1). This is preferably automatically performed by the software. Additionally, a standard can be measured in conjunction with the measuring of the sample. The concentration of this standard is likewise known and input into the program. The Cp value and the concentration of the simultaneously measured standard are compared to the standard curve, and the concentration of the unknown sample is calculated using these data and the Cp value of the sample.

The standard for generating a standard curve is a nucleic acid of the organism for which it is assumed that the biological material obtained or extracted from or produced by the organism contains a nucleic acid of the organism in question. An “organism” can be a prokaryote (e.g., bacteria) or a eukaryote (e.g., mammal, bird, fish, reptile, insect, fungus (including yeast), a virus, or an archaeon. A preferred organism is a host cell as defined herein, which produces biological material or from which biological material is obtained or extracted.

In real-time PCR nowadays, calculations are no longer made primarily in terms of DNA product quantities or concentrations; instead the so-called Ct or Cp (=crossing point) values are used as a measurement for quantifying the starting quantity. These values correspond to the number of PCR cycles that are necessary to achieve a constant, defined fluorescence level. At the Cp, all reaction vessels contain the same quantity of newly synthesized DNA. In the case of 100% efficiency of the PCR, the DNA product quantity, and in an analogous manner the fluorescence signal, doubles with each cycle. A Cp that is lower by one unit corresponds to twice the cDNA used; or rather the mRNA starting quantity.

If for example SYBR Green or another dye intercalating in double-stranded DNA is used for the qPCR, a melting curve can be recorded immediately following the qPCR. This gives information on the purity of the amplicons. For the melting curve, the temperature is raised continuously immediately after the last cycle (usually from 45° C. to 95° C.). The fluorescence signal is measured continuously in this process. More and more DNA denatures due to this slow increasing of the temperature. If the DNA is denatured, SYBR Green can no longer bind to it and the fluorescence diminishes. The time of the DNA denaturing is determined chiefly by the GC content and the length of the DNA. As a result, each PCR product has its own melting point. Because the amplicons are contained in the sample in large numbers, the fluorescence diminishes abruptly at the melting temperature of these amplicons (see FIG. 2). To depict the melting curve, fluorescence is plotted against temperature. The first derivation of fluorescence as a function of temperature can be depicted for simple analysis. The melting points thus become visible (compare the modes of depiction in FIG. 2). Ideally there is only one melting point with a PCR. If there are several, there can be diverse reasons for this. These include, for example, primer dimer formation or a non-specific binding of the primers. The melting curve analysis represents an important method for the optimization of a qPCR.

The lower limit of the method of the invention for detecting a nucleic acid, preferably double-stranded DNA, is between 10 fg-0.08 fg/μl, by way of example 10, 9, 8, 7, 6, 5, 4, 3, 2, 1, 0.5, 0.4, 0.25, 0.1, 0.09 or 0.08 fg/μl, for example. The quantification is preferably effected absolutely, i.e. as absolute quantification.

A sample that contains biological material is then advantageously deemed negative for a nucleic acid (i.e., it does not contain any detectable nucleic acid) if the Cp value of the sample to be measured corresponds to the Cp value of the standard curve at which the Cp value does not exceed the Cp value of the background with the method according to the invention, in other words is equal to or even less than the background value. By “background”, it is meant that a sample to be measured does not contain any nucleic acid. This can be achieved by adding, for example, nucleases such as DNases, RNases, or acid to such a negative sample.

The sample can be liquid or solid, wherein a solid sample is preferably liquefied, for example by being dissolved or suspended in an aqueous medium. The sample can be any liquid or solid that contains biological material. Liquids can be, for example, body fluid of a mammal, bird, fish, reptile, or insect, but also the cytosol or the cell wall or cell membrane of mammal cells, bacteria cells, fungus cells including yeast cells, fish cells, bird cells, reptile cells, viruses, or insect cells. Examples of body fluids include sputum, secretions, urine, blood, serum, plasma, sperm, cerebrospinal fluid, breast milk, tear fluid, etc. Fluids can furthermore be the fermentation broth or the supernatant of a culture of the aforementioned cells, in particular the fermentation broth or the cell culture supernatant of production systems known per se or newly developed for biologically and/or biosynthetically produced medications (e.g. CHO cells, E. coli cells, Bacillus subtilis cells; insect cells, yeast cells, etc.). The sample is preferably a biopharmaceutical or biotechnological product.

The biological material of the sample, which is fed into a process according to the invention for detecting a nucleic acid in a sample, preferably comes from mammal cells, bacteria cells, or fungus cells, but can also come from fish cells, bird cells, reptile cells, viruses, or insect cells. For the purposes of the invention, mammal cells preferably include cells of humans, mice, hamsters, rats, rabbits, camels, llamas, dogs, cats, horses, cows, or pigs.

The biological material can contain nucleic acids such as DNA (for example genomic DNA, plasmid DNA, DNA from organelles), RNA (for example mRNA, rRNA, miRNA, siRNA, and/or tRNA), proteins, carbohydrates, and/or lipids, etc. Nucleic acids of the biological material, preferably DNA or RNA as described above and elsewhere (single-stranded—sometimes abbreviated “ss” as well as double-stranded—sometimes abbreviated “ds”), are detected in the biological material with the method according to the invention, whereas proteins, carbohydrates, and/or lipids are preferably not detected.

The biological material is preferably intended to be administered to animals or human beings. Preference is given to administration to human beings. Particular preference is given to the intravenous administration of the biological material to humans or animals. It is of particular interest not to administer any nucleic acid contained in the sample to humans. This is particularly important because biotechnologically produced products for therapy, diagnosis, cosmetics, or foods often come from host cells, e.g., also from mammal cells such as human cells/cell lines, and it is desirable not to administer any nucleic acids of these host cells, or else to administer nucleic acids only in quantities within the limits approved by authorities (unavoidable) to humans. The method according to the invention now makes it possible to detect such nucleic acids in a sample, in order to test the biological material (intended for administration to humans) of the sample with sufficient sensitivity such that potential hazards or undesired side effects associated with the administration can be avoided or even excluded at the outset as much as possible. This is possible because the method according to the invention surpasses the previous lower limits for the concentration of nucleic acids in biological material such that, due to the higher sensitivity of the method according to the invention, a higher assurance of safety can be established regarding the quantity of nucleic acid (of a host cell used for the production) in the biological material.

The nucleic acids contained in the biological material can be DNA or RNA, double-stranded or single-stranded. The DNA can be genomic DNA, plasmid DNA, cosmid DNA, bacmid DNA, etc.; the DNA is preferably genomic DNA. In the case of the detection of RNA, a reverse transcription is advantageously carried out in order to convert RNA into cDNA. In other words, in the case of detection of RNA in a sample that contains biological material, a reverse transcription is advantageously carried out before the treatment with protease, but ultimately RNA is reverse transcribed into cDNA prior to step (d) at the latest, which in turn means that the reverse transcription can be carried out before or after one of steps (a) through (c). In other words, the method according to the invention can also include the step of subjecting the sample to a reverse transcription in addition to the aforementioned steps (a) through (d). For the reverse transcription, use is made of either oligo-dT primers or random 6-mer, 8-mer or 10-mer primers together with the enzyme reverse transcriptase (RT). If the nucleic acid comes from eukaryotes, oligo-dT primers are preferably used for the reverse transcription. The enzyme RT is preferably inactivated after the reverse transcription.

The biological material of a sample contains at most nucleic acid, preferably DNA. In other words, a method according to the invention preferably relates to the detection of a nucleic acid in a sample that contains biological material, wherein the biological material contains no more than 1 ng (=1000 pg), preferably no more than 500 pg, 400 pg, 300 pg, 200 pg, 100 pg, 50 pg, 25 pg, or 10 pg per dose. A dose can be 1, 5, 10, 20, 30, 40, 50, 60, 70, 80, 90, 100, 150, 200, 250, or more milliliters.

The nucleic acids to be detected with the method according to the invention can come from a host cell, which represents the biological material per se or produces the biological material. In the last case mentioned, the nucleic acids contained in the biological material originating from (produced by) the host cell ultimately come from the host cell and should be detected with the method of the invention, provided that any detectable nucleic acid is contained in the sample in the first place. Such host cells are described in more detail herein.

As stated above, a preferred embodiment of this invention is one in which biological material, in particular biotechnologically produced material, is tested for the possible presence of nucleic acids originating from, for example, host cells that are used in the biotechnological production. For the purposes of the invention, host cells can be: mammal cells, bacteria cells, fungus cells, fish cells, bird cells, reptile cells, insect cells, or viruses, for example. The biological material can contain antibodies or a protein (for example, a therapeutic protein), which is/was produced by a host cell in particular.

For the purposes of the invention, preferred mammal cells are PER.C6, HEK cells, primate cells, e.g. Vero cells, NS0 cells, CHO cells, for example DUXB11, DG44, or CHOK1, mouse hybridoma cells, rat hybridoma cells, or rabbit hybridoma cells.

The expression “antibody” includes any antibody, derivatives, or functional fragments thereof that still have their binding specificity. Methods for producing antibodies are sufficiently known and described in the field, for example in Harlow and Lane “Antibodies, A Laboratory Manual”, Cold Spring Harbor Laboratory Press, 1988, and Harlow and Lane “Using Antibodies: A Laboratory Manual” Cold Spring Harbor Laboratory Press, 1999. The expression “antibody” also includes immunoglobulins (Igs) of various classes (i.e., IgA, IgG, IgM, IgD, and IgE) and subclasses (such as IgG1, IgG2, etc.) as well as molecules derived therefrom. These antibodies can be used for, e.g., immunoprecipitation, affinity clean-up, and immunolocalization of polypeptides or fusion proteins of the invention, as well as for monitoring the presence and the quantity of such polypeptides, for example in cultures of recombinant prokaryotes or of eukaryotic cells or of organisms. The definition of the expression “antibody” furthermore includes embodiments such as chimeras, single-chain and humanized as well as human antibodies, and also antibody fragments such as Fab fragments, etc. Antibody fragments or derivatives furthermore include F(ab), F(ab)₂, F(ab′)₂, Fv, scFv fragments or antibodies with a single domain, e.g., nanobodies or domain antibodies, antibodies with a single variable domain or a single variable domain of immunoglobulin that comprises only one variable domain, which can be VH or VL, which specifically binds an antigen or epitope independently of other V regions or domains (see for example Harlow and Lane (1988) and (1999), loc. cit.). Such individual variable domains of immunoglobulins comprise not only a polypeptide of an isolated antibody with a single variable domain, but also larger polypeptides that comprise one or several monomers of a polypeptide sequence of an isolated antibody with a single variable domain. Antibody fragments or derivatives furthermore comprise bispecific antibodies, for example bispecific single chain antibodies (scFv), diabodies, tetrabodies, or DART antibodies. Various methods are known in the field and can be used for producing such antibodies and/or fragments. Hence the (antibody) derivatives can be produced using peptide mimics. Furthermore, methods described for the production of single chain antibodies (see for example U.S. Pat. No. 4,946,778) can be adapted in such a way that they produce single chain antibodies that are specific for one or several selected polypeptides. Furthermore, transgenic animals can be used to express humanized antibodies that are specific for polypeptides and fusion proteins of this invention. To produce monoclonal antibodies, use can be made of any method that provides antibodies that are produced by continuous cell line cultures. Examples of such methods include the hybridoma method (Köhler and Milstein, Nature 256 (1975, 494-497), the trioma method, the human B cell hybridoma method (Kozbor, Immunology Today 4 (1983), 72), and the EBV hybridoma method for producing human monoclonal antibodies (Cole et al., Monoclonal Antibodies and Cancer Therapy, Alan R. Liss, Inc. (1985), 77-96). A surface plasmon resonance, as used in the BIAcore system, can be employed to increase the efficiency of phage antibodies that bind to an epitope of a target polypeptide, e.g., CD3 epsilon (Schier, Human Antibodies Hybridomas 7 (1996), 97-105; Malmborg, J. Immunol. Methods 183 (1995), 7-13). In the context of this invention, the expression “antibody” shall furthermore be considered to include antibody constructs that can be expressed in a host as described below, for example antibody constructs that can be transfected and/or transduced by viruses or plasmid vectors, etc.

An antibody that is contained in the biological material according to the invention is preferably an antibody against EGFR, Her2, TA-MUC1, TF, or LeY.

Examples of preferred antibodies are the anti-EGFR antibody Cetu-GEX®, the anti-Her2 antibody TrasGEX®, the anti-TA-MUC1 antibody PankoMab-GEX® or SeeloMab-GEX®, the anti-TF antibody GatoMab-GEX®, Karomab-GEX® or Teltomab-GEX® or the anti-LeY antibody LindoMab-GEX®.

A therapeutic protein that is contained in the biological material according to the invention is preferably a hormone, enzyme, or cytokine, or a protein or peptide derived therefrom.

Examples of a preferred therapeutic protein include the hormones FSH, hCG, hLH, or hGH. Further examples of a preferred therapeutic protein include the clotting factors Factor VII, Factor FVIIa, Factor FVIII, Factor VIIIa, Factor IX, Factor IXa, Factor X or Factor Xa, or fusion proteins and derivatives with/from these proteins. Still further examples of a preferred therapeutic protein include enzymes that are used as part of an enzyme replacement therapy (ERT), for example glucocerebrosidase or galactosidase, or fusion proteins and derivatives with/from these proteins. ERT is a therapeutic technique for treating enzyme defects associated with lysosomal storage diseases. In this technique, recombinant enzymes are administered to patients via infusion or injection.

The protease used in step (a) of the method according to the invention can basically be any protease, with preference being given to using a non-specific serine protease (e.g., proteinase K). Such a protease has a very broad recognition spectrum. It cuts the carboxyl ends of aromatic, hydrophilic, and aliphatic amino acids. In this process, proteinase K splits proteins in the following manner: X-↓-Y-, wherein X represents an aliphatic, aromatic, or hydrophobic amino acid, and Y represents any amino acid. Proteinase K is activated by denaturing substances such as SDS. In the method according to the invention, use is made of a protease, preferably proteinase K, to remove proteins present in the biological material of a sample. A protease that is used in the method according to the invention is advantageously free of contaminating nucleic acids, in particular free of DNA.

Persons skilled in the art are aware of the incubation times of a sample with a protease. They will preferably treat the sample with the protease for at least 1 hour or longer, for example 2, 3, 4, 5, 6, 7, 8, 9, 10, 12, 18 or 24 hours, e.g., overnight. Persons skilled in the art are also aware of the temperature at which the sample with protease is to be treated; for this purpose persons skilled in the art will preferably use a temperature of between 37° C.-53° C.; persons skilled in the art may optionally add SDS to the denaturing formulation. Persons skilled in the art are likewise familiar with the quantities of protease to use; however, they will preferably use between 1-5 mg/ml, e.g., 2 mg/ml. For the case in which the protease is to be inactivated, provision is made of, for example, an inactivation at 95° C. for a period of at least 10 minutes or longer.

The concentration of a sample in step (b) of the method according to the invention is preferably carried out by means of filtration with volume reduction.

Substances present in a solution are separated out during a filtration. In this process, the membrane represents a barrier that is either permeable to or retains substances, based on its physical or chemical properties. Membranes are normally classified on the basis of their pore size. Ultrafiltration is a preferred filtration that is used with the method according to the invention. In ultrafiltration, the pore size is 0.001-0.1 μm on average. The membranes retain molecules with molecular weights of 300 to 10,000,000 Da (Dalton).
Preference is given to using membranes according to the invention with a 10k filter, for example Amicon ultrafiltration units with 10K filters. This means that the latter have a NMWL (Nominal Molecular Weight Limit) of 10,000 Da. Molecules with molecular weights less than 10 kDa (kilodalton) can pass through the membrane. Heavier molecules cannot.
The sample is pressed by centrifugation through the nearly vertically aligned membrane (which is composed of regenerated cellulose) and concentrated from several milliliters down to a few microliters. A sample is preferably concentrated by a factor of 10-20 in step (b) of the method according to the invention.

An Amicon Ultra-4 centrifugal filter unit (10 K) is a preferred device for carrying out step (b) of the method according to the invention.

The enrichment of the nucleic acid in step (c) of the method according to the invention is preferably effected by affinity chromatography or precipitation. The affinity chromatography is preferably effected by silica adsorption or adsorption on polymer particles. The silica adsorption is preferably effected by means of a silica-based membrane.

A common principle in nucleic acid enrichment is based on affinity chromatography, in particular on a silica membrane. A silica membrane is composed of quartz (SiO₂), which is bound to OH⁻⁻ groups. A nucleic acid is dissolved in water, for example. The nucleic acid as well as the silica membrane are surrounded by a hydrate shell. Chaotropic salts are composed of ions that reduce hydrophobic effects. Examples of such ions include SCN⁻, H₂PO₄⁻⁻; NH₄⁺, K⁺, or guanidine. Adding chaotropic ions destabilizes the hydrate shell of the membrane and of the DNA, and the DNA binds to the silica membrane. It is hypothesized that intermolecular hydrogen bridge bonds form between the backbone of the DNA and the OH⁻ group of the membrane. The DNA is adsorbed onto the membrane by these bonds. Another hypothesis describes a saturation of the negative OH groups with positively charged ions. These positive ions form a cation bridge to the backbone of the DNA. The nucleic acid is thus bound firmly to the silica membrane and can be washed. Solutions with high concentrations of chaotropic salts or ethanol should preferably be chosen as wash/elution buffers.

It should be noted that in step (c) of the method according to the invention, preference is given to carrying out the same procedure as for isolating a nucleic acid. In other words, it is not assumed at the outset that a nucleic acid is present in free form, i.e. in solution, in the sample that contains biological material. For example, the instructions of a commercial kit, as described below, are followed.

Preferred examples of polymer particles are polystyrene particles, e.g., Dynabeads.

A precipitation of nucleic acids, in particular DNA, is carried out in accordance with methods known per se.

The inventors found that the yield of nucleic acids, in particular of DNA, was better if the elution or washing-off of the silica membrane with wash/elution buffer was carried out at 45° C. rather than at room temperature (19-26° C.). Hence eluting the nucleic acid, in particular DNA, from the silica-based membrane in step (c) at 45° C. is a preferred embodiment of the method according to the invention.

Examples of devices for enriching the nucleic acid in step (c) of the method according to the invention include the NucleoSpin® Plasma XS Kit, QIAamp UCP Pathogen Mini Kit, QIAamp DNA Investigator Kit, QIAamp Viral RNA Mini Kit, NucleoSpin® Tissue XS Kit, NucleoSpin® Trace Kit, nexttec clean Column, DNA Extraction EZ-Kit, forensicGEM Tissue Kit, with preference being given to the QIAamp DNA Investigator Kit.

The detection of the nucleic acid by means of quantitative PCR (qPCR) in step (d) of the method according to the invention preferably focuses on nucleic acids that a person skilled in the art would expect in the sample that contains biological material. For example, in the case of a sample that contains biological material that was produced biotechnologically by means of a host cell, he would focus on nucleic acids that would possibly be contained by the host cell in the sample. That means that, in a preferred embodiment, the focus in step (d) of the method according to the invention will be on nucleic acids, in particular on DNA, of the host cell that was used for the biotechnological production of the biological material. For example, the focus would be on E. coli DNA in the event that E. coli was used, on yeast DNA in the event that yeast was used, on CHO DNA in the event that CHO cells were used, or on human DNA in the event that a human cell was used. Naturally persons skilled in the art know that, when using one of the host cells described herein for the production of biological material, in step (d) they will focus on nucleic acids, in particular DNA, of the host cells described herein. In the case of human DNA, the focus is preferably on repetitive elements, preferably on repetitive elements in mammal DNA. Repetitive elements can preferably be Alu sequences or Alu-equivalent sequences.

The standard necessary for the detection of the nucleic acid is one such as defined above. Accordingly, step (d) preferably relates to detecting the nucleic acid by means of quantitative PCR, the quantitative PCR focusing on nucleic acids that persons skilled in the art would expect in the sample that contains biological material, as described in more detail above.

In step (d) of the method according to the invention, the primer pair preferably has SEQ ID No. 1 and SEQ ID No. 2. Another particularly preferred primer pair has SEQ ID No. 3 and SEQ ID No. 4. Other particularly preferred primer pairs are ones with SEQ ID Nos. 5 and 6. Additional preferred primer pairs are ones with SEQ ID Nos. 1 and 7, 1 and 8, 1 and 9, 1 and 10, or 11 and 12 (also see FIG. 5). Also preferred is a mixture of the primer pairs with SEQ ID Nos. 1 and 7-10.

Eukaryotes bear different types of mobile DNA elements in their genomes, which occur in different numbers and can be classified on the basis of sequence homologies. More than 45% of the human genome is composed of mobile elements. The Alu sequences form one of the largest groups of mobile elements. These are distributed in high copy number, i.e. repetitively, and uniformly over the human genome. Within the genome there are different kinds of repetitive sequences. The copies can be present directly adjacent to one another in large number as “tandem repeats”, as in the telomere zone of chromosomes, for example. Alu elements are so-called SINEs (short interspersed elements). SINEs are distributed over the entire genome and have a maximum size of 500 bp (base pairs). Alu elements are small sequences 300 bp in size, which are repeated many times (more than a million copies) in the genome. Alu sequences thus represent not only the largest amount of mobile elements, but also the largest amount of SINEs. Because of this large number of copies, Alu sequences represent more than 10% of the human genome mass.

A qPCR based on the Yb8 Alu subfamily is described in the publication entitled “Human DNA quantitation using Alu element-based polymerase chain reaction” by J. A. Walker et al. (2003), Anal. Biochem. 315, 122-128. Due to the human genome specificity of the Yb8 Alu elements, the high copy number of 1852, and the fact that these elements are distributed in the entire genome, these Alu sequences are especially well-suited for the specific quantification of human DNA. The use of primer pairs that focus on the Yb8 Alu subfamily is a preferred embodiment for the purposes of this invention. A particularly preferred primer pair has SEQ ID No. 1 and SEQ ID No. 2. Another particularly preferred primer pair has SEQ ID No. 3 and SEQ ID No. 4. Other particularly preferred primer pairs are ones with SEQ ID Nos. 5 and 6. Additional preferred primer pairs are ones with SEQ ID Nos. 1 and 7, 1 and 8, 1 and 9, 1 and 10, or 11 and 12. Also preferred is a mixture of the primer pairs with SEQ ID Nos. 1 and 7-10.

Repetitive sequences not found exclusively in human DNA, but also in other genomes. Several repetitive elements that show a significant homology to the human Alu sequences have been found in CHO (Chinese Hamster Ovary) cells, hence they are called CHO Alu-equivalent elements. Due to their specific sequence and their high copy number, these CHO Alu-equivalent sequences are well-suited as primers for the quantification of CHO DNA. The use of primer pairs that focus on CHO Alu-equivalent elements is therefore a preferred embodiment for the purposes of this invention. A particularly preferred primer pair has the sequences 5′-TGGAGAGATGGCTCGAGGTT-3′ (SEQ ID No. 5) and/or 5′-TGGTTGCTGGGAATTGAACTC-3′ (SEQ ID No. 6).

This invention further relates to a kit for carrying out a method according to the invention, said kit comprising
(a) Protease, preferably Proteinase K;
(b) Means for concentrating liquid samples, preferably a device for an ultrafiltration with volume reduction;
(c) Means for the affinity chromatography, preferably a device that employs silica adsorption; and
(d) DNA polymerase and a primer pair for amplifying repetitive elements in DNA, preferably human DNA.
   Embodiments described in conjunction with the method according to the invention also apply, mutatis mutandis, to the components (a) through (d) of the kit according to the invention.

### FIGURES

FIG. 1: Principle of the absolute quantification of DNA by means of qPCR

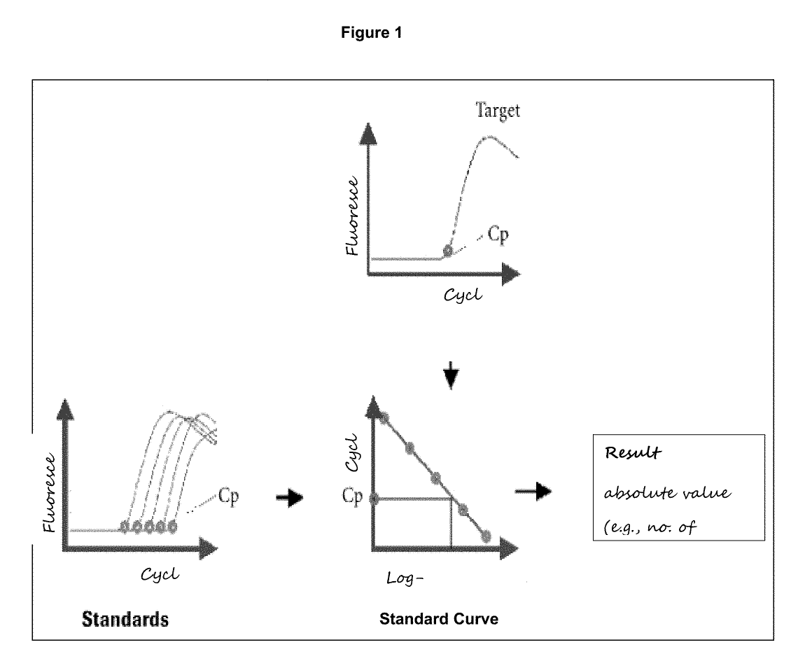

FIG. 2: Melting curve and melting points (see text for explanations) of a specific and non-specific qPCR. Figure from Roche 2008.⁶ The green curve is a specific qPCR, on which it can be discerned that there is only a steep drop of the melting curve and therefore only one melting point. The blue curve in contrast is non-specific, which can be discerned from the two maxima.

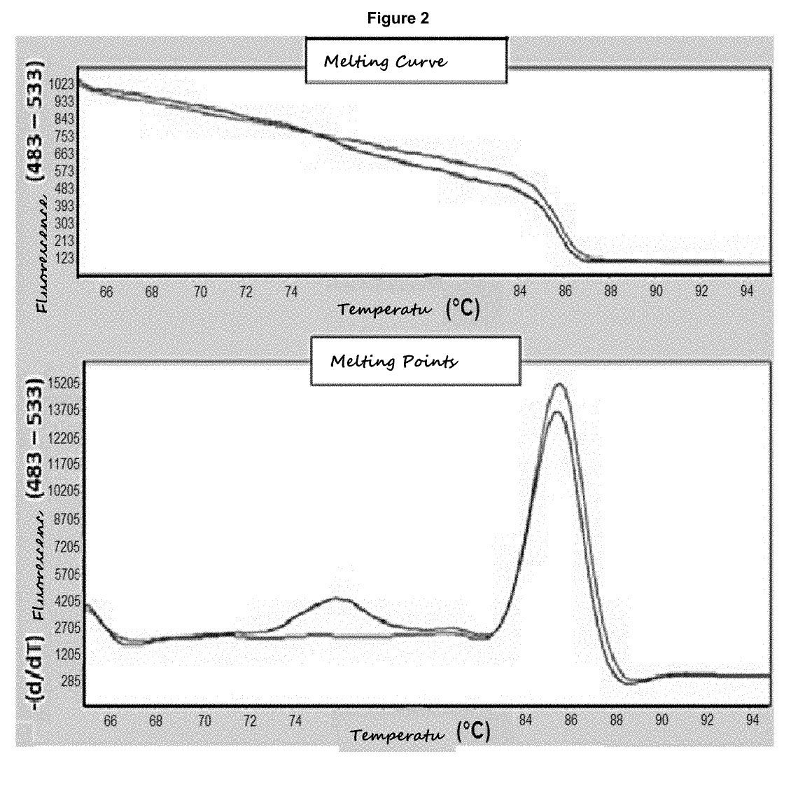

FIG. 3: E1-DNA standard line, including standard deviation The associated measurement values can be determined from Table 11; red measurement points are measurement values of the E1-DNA, blue measurement points are those of the negative control (blue measurement point=point of intersection with the zero point of the y-axis).

FIG. 4: Standard line recorded and stored on the LightCycler480 for determining human DNA concentrations.

FIG. 5: Alu primer sequences of SEQ ID Nos. 1 and 11 as well as 2 and 12. Shown are possible permutations of the forward primer (top sequence) and of the reverse primer (bottom sequence), which are reflected in SEQ ID Nos. 11 and 12, respectively.

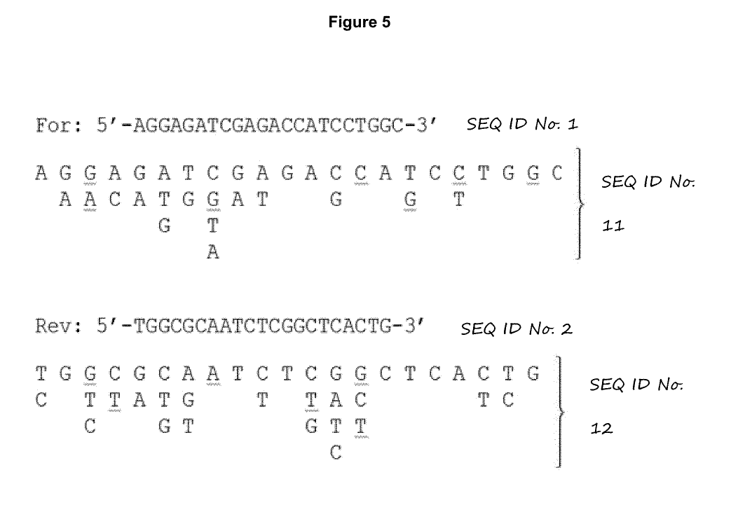

### EXAMPLES

### Example 1

1. Performance of Human qPCR with Yb8 Primers
   The master mix for all samples was prepared first. The final concentration of the forward primer (SEQ ID No. 3) is 0.15 μM; that of the reverse primer (SEQ ID No. 4) is 0.2 μM. The PCR master mix was thoroughly mixed by repeatedly tapping on the tube. To perform the negative controls, in each case 20 μl PCR master mix were pipetted into five wells, which were immediately sealed with a PCR cover. Using the Xstream multipipette, 17 μl PCR master mix were prepipetted into each necessary well of the MWP (multi-well plate). 3 μl sample were added afterwards. The MWP was tightly sealed with film with the aid of the scraper, spun down at 1500 rpm for 2 minutes, and placed directly in the LightCycler. The PCR run was then started under the following conditions:

| | | | Temperature |
|---|---|---|---|
| Step | Temperature | Time | decrease/increase |
| Denaturation | 95° C. | 5 s | 4.4° C./s |
| Annealing + Elongation | 71° C. | 20 s | 4.4° C./s |

2. Preparation of CetuGEX™ Samples for Eventual Quantification of Residual Human DNA by Means of qPCR
   As already mentioned, according to FDA regulations, for example, the quantity of residual host cell DNA may not exceed 100 pg/dose. Because provision is made for a 200 ml dose with CetuGEX™, the amount of antibody-producing host cell DNA may not exceed 0.5 pg/ml. This low DNA concentration could not be measured by using human qPCR alone. The inventors therefore found a way to remedy this problem and provided the method according to the invention.
   The samples were prepared in 3 steps: as a first step, a proteinase K digestion was carried out, in which the proteins that are present in large quantities in CetuGEX™ were removed in order to prepare for the following step. In the second step, the DNA was concentrated using ultrafiltration. Without the pretreatment with Proteinase K, this solution would have been too viscous. Because they might have contained PCR-inhibiting substances, the concentrated samples were purified in the final step. This purification was performed with a commercially available kit.
2.1 Proteinase K digestion of CetuGEX™
   40 U protease K and 40 pl 10% SDS solution were added to 4 ml sample and thoroughly mixed. This mixture was incubated at 53° C. overnight. The 15 ml tube was tightly sealed with Parafilm to prevent contamination.
2.2 Ultrafiltration of CetuGEX™ for Concentrating Residual Human DNA
   After being digested with Proteinase K, the sample was centrifuged for 2 min (4000 rcf) and placed in an Amicon ultrafiltration unit. The Amicon ultrafiltration unit was centrifuged for 8 min at 4000 rcf (Hettich centrifuge) in the next step. The supernatant in the filter was removed and transferred into 2 ml tubes, centrifuged for 5 min at 12,300 rcf in the tabletop centrifuge, and the volume was determined using the Xstream multipipette. This volume formed the basis for calculating the water volume for the subsequent washing step. The preparation volume at this stage should be a total of 400 μl per sample, in other words the volume of the ultrafiltrate plus the washing volume. Washing was accordingly performed with the volume obtained by subtracting the ultrafiltrate volume from 400 μl. The washing step was carried out with PCR grade water. Each side of the filter was washed 7 times, and this wash volume was added to the ultrafiltrate.
2.3 Clean-Up of the Concentrated CetuGEX™ Samples for the Measurement of Human DNA by Means of qPCR
   Different DNA clean-up methods, which are each based on the kit mentioned in the title, shall be described in the following sections. Unless stated otherwise, these were small-scale preparations for which a tabletop centrifuge (VWR®) could be used.
2.3.1 NucleoSpin® Plasma XS Kit
   After the sample was treated by Proteinase K digestion and ultrafiltration, 600 μl BB buffer were added to 400 μl thereof. The tube was inverted three times, vortexed for 3 sec, and then briefly spun down. The subsequent preparation steps could be carried out using the VWR tabletop centrifuge. 500 μl of the mixture in each case were applied two times to the column contained in the kit, centrifuged at 2,000×g for 30 sec, and the collection vessel was discarded. After another centrifugation step at 11,000×g for 5 sec, 500 μl wash buffer (WB) were added to the column. The column was then centrifuged at 11,000×g for 30 sec, washed again with 250 μl WB, and centrifuged for 3 min at 11,000×g. The column was transferred to a DNA-free Eppendorf tube, 40 μl elution buffer were added, and the tube with the column was centrifuged at 11,000×g for 30 sec.
2.3.2 QIAamp DNA Investigator Kit
   After the sample was treated by Proteinase K digestion and ultrafiltration, 40 μl AW1 buffer and 1 ml AW2 buffer were admixed with 400 μl of the treated sample. The mixture was mixed for 10 sec with a vortexer, and 720 μl thereof were applied to a QIAamp MinElute column. After centrifugation at 6,000×g for 1 min, the collection vessel was changed and the remaining sample was added. After centrifugation again under the same conditions and changing of the collection vessel, 500 μl of the AW2 Puffers were applied to the column and centrifuged at 6,300×g for 1 min. After changing the collection vessel again, the membranous, DNA-binding matrix was freed of excess fluid by centrifugation at maximum speed (12,300×g) for 3 min. The column was placed for elution in a DNA-free Eppendorf tube, 40 μl ATE buffer were pipetted onto the center of the membrane, and it was incubated for 10 min at room temperature. After centrifugation at 12,300×g for 1 min, on average ca. 38 μl eluted DNA were present in the Eppendorf tube.
2.3.3 QIAamp UCP Pathogen Mini Kit
   400 μl of sample and 200 μl APL2 were mixed for 30 sec. After adding 300 μl ethanol, the mixture was vortexed for another 30 sec. 600 μl of the mixture were applied to the QIAamp UCP Mini Spin column and centrifuged at 6,000×g for 30 sec (VWR® tabletop centrifuge). After changing the collection vessel, the rest of the mixture was pipetted onto the column and centrifuged again under the same conditions. The collection vessel was changed again and 600 μl APW1 buffer were added to the column. The collection vessel was changed after centrifugation at 6,000×g for 1 min. The column was centrifuged at 13,200×g for 3 min after 750 μl APW2 buffer were added to it. To dehumidify the membrane, the column was placed in a new collection vessel, centrifuged at 12,300×g for 1 min, and the column was then incubated, with the cover open, in a heating block at 56° C. for 3 min. The column was then placed in a DNA-free Eppendorf tube and, after addition of 40 μl AVE buffer, incubation for 1 min, and centrifugation at 12,300×g, the eluted DNA was present in the tube.
2.3.4 NucleoSpin® Tissue XS
   After the sample was treated by Proteinase K digestion and ultrafiltered, 400 μl B3 buffer and the same amount of ethanol were admixed with 400 μl of the sample. This mixture was mixed and 400 μl thereof were added to the column furnished with the kit. The column was centrifuged at 11,000×g for one minute, the collection vessel was changed, and in two further steps, the remaining mixture was applied to the column and centrifuged. After the collection vessel was changed again, a volume of 50 μl B5 buffer was added to the column for washing, followed by centrifugation at 11,000×g for one minute. The discharge was discarded and the column was washed a second time with another 50 μl of B5 buffer. The column was centrifuged at 11,000×g once again. The column was placed in a DNA-free Eppendorf tube and 40 μl BE buffer were added to the column. The eluted DNA was transferred into the Eppendorf tube by centrifugation at 11,000×g for one minute.
2.3.5 NucleoSpin® Trace Kit
   Due to the large volumes, this method had to be carried out in the Hettich centrifuge. In contrast to the preceding DNA preparation methods, an upstream ultrafiltration step was not required here. After 4 ml of sample were treated by Proteinase K digestion, 8 ml FLB and 3.5 ml ethanol were added to this volume. The mixture was thoroughly mixed and applied to the column of the kit. After centrifugation at 3,000×g for 3 min, the collection vessel was replaced with a new one and the column was washed three times. The washing steps were carried out once with 2.5 ml BW and twice with 5 ml B5 buffer. After addition of the respective wash buffers, the mixture was centrifuged each time for 3 min at 3,000×g. After changing the collection vessel once again, the silica membrane was freed of excess liquid by centrifuging for 10 minutes at 3,000×g. After the 1.5 ml tube included in the kit was set on the column and both were placed together in a 50 ml tube for centrifugation, 100 μl BE buffer were applied to the silica membrane in order to induce the elution. After two minutes of reaction time at room temperature, the construction was centrifuged at 3,000×g for 3 min. The eluted DNA was present in the 1.5 ml tube.
2.3.6 Nexttec clean Columns
   The Nexttec clean Columns have to be equilibrated before they can be used. To do so, 350 μl preparation solution (i.e., the elution solution for DNA used previously) were applied to the columns, which were incubated for at least 5 min at room temperature. The solution was then spun down for 1 min at 350×g. The column was placed in a DNA-free Eppendorf tube. Next 100 μl of sample Probe were applied to the column, incubated for 3 min at room temperature, and centrifuged at 700×g for 1 min. This method is thus a gel filtration method that is not based on a DNA-binding matrix.
2.3.7 DNA Extraction EZ-Kit
   After the sample was treated by proteinase K digestion and ultrafiltered, 20 μl Detergent Combo solution were added to 500 μl of the sample and mixed briefly. This mixture was incubated for 10 min at 60° C. 1 μl glycogen was mixed with 500 μl sodium iodide, and this mixture was added to the sample mixture. After vortexing again, it was incubated again at 60° C. for 10 min. To precipitate the DNA, 900 μl isopropanol were then added to the mixture, which was vortexed and incubated at room temperature for 30 min. The mixture was centrifuged at 12,000 rpm for 10 min and the supernatant was carefully poured off. 1.8 ml wash buffer were added to the DNA pellet, and then the tube was vortexed. For pelleting, the mixture was centrifuged for another 10 min at 12,000 rpm. The supernatant was carefully poured off and the pellet was air-dried for 1 h. The pellet was resuspended with 50 μl PCR grade water.
2.3.8 forensicGEM Tissue Kit
   4 ml of undigested sample were placed in an Amicon ultrafiltration unit and concentrated by centrifugation at 4,000 rpm in the Hettich centrifuge down to ca. 50 μl (duration ca. 50 min). The volume of the sample was determined, and each membrane of the ultrafiltration unit was washed 7× with sufficient water for a total volume of wash water plus sample equal to 89 μl. A mixing process and a brief centrifugation followed. After addition of 10 μl 10×buffer and 1 μl prepGEM, the mixture was incubated, first for 15 min at 75° C. and immediately afterwards for 5 min at 95° C. This solution could be used directly for qPCR (see 3.4.1).
2.3.9 Testing of Human DNA-Free Kits with CetuGEX™
   After it was proven that four of the kits were contamination-free, these kits were tested with CetuGEX™ and compared to each other. It can be discerned in Table 1 that the lowest possible Cp values were achievable by using the NucleoSpin® Tissue Kit and the QIAamp DNA Investigator Kit for clean-up.

TABLE 1
Mean Cp values of the CetuGEX qPCR, with clean-up by various kits.

| | | CetuGEX ™ + |
|---|---|---|
| | CetuGEX ™ | 2 pg human DNA |
| forensicGEM Tissue Kit | — | — |
| NucleoSpin ® Trace Kit | 38.23 | 36.12 |
| NucleoSpin ® Tissue XS Kit | 34.44 | 31.35 |
| QIAamp DNA Investigator Kit | 34.63 | 32.15 |

2.3.9.1 Testing of the QIAamp DNA Investigator Kit and of the NucleoSpin® Tissue Kit with CetuGEX™ and Different DNA Standard Concentrations
   After narrowing down the kits to the two with the best preliminary results, a final experiment was performed for the direct comparison of the NucleoSpin® Tissue XS Kit and the QIAamp DNA Investigator Kit. CetuGEX™, partly additioned with standards, was used as the sample. Three columns were tested with each sample, and each column was measured in triplicate. The individual Cp values can be seen. Table 2 shows the mean values of the individual columns and the mean values of the three columns of a sample.

TABLE 2
Mean Cp values of the NucleoSpin ® Tissue XS Kit and QIAamp DNA Investigator Kit
comparison test. CetuGEX ™ formulations without the addition of standard, and with the
addition of 2 pg, 20 pg, and 200 pg DNA per column were measured; “difference”: see text.

| | Addition of | | | | |
|---|---|---|---|---|---|
| Kit | human DNA | — | 2 pg | 20 pg | 200 pg |
| NucleoSpin ® | Ø Cp Sample 1 | 32.47 | 30.93 | 27.23 | 24.54 |
| Tissue XS Kit | Ø Cp Sample 2 | 35.05 | 34.73 | 31.78 | 28.53 |
| | Ø Cp Sample 3 | 35.81 | 34.87 | 33.48 | 28.15 |
| | Overall Ø | 34.44 | 33.51 | 30.83 | 27.07 |
| QIAamp DNA | Ø Cp Sample 1 | 31.19 | 29.64 | 27.09 | 23.72 |
| Investigator Kit | Ø Cp Sample 2 | 36.17 | 32.94 | 32.09 | 27.82 |
| | Ø Cp Sample 3 | 34.40 | 32.36 | 31.61 | 29.45 |
| | Overall Ø | 33.92 | 31.65 | 30.26 | 27.00 |
| Difference | | 0.52 | 2.18 | 0.56 | 0.07 |

It can be discerned that the QIAamp DNA Investigator Kit has lower Cp values in the overall comparison. This becomes even clearer in the “Difference” column, in which the following was calculated:

Overall Ø (NucleoSpin® Tissue XS Kit)−Overall Ø (QIAamp DNA Investigator Kit)

The positive values in the “Difference” column confirm that, in terms of the mean of all Cp values of a sample (e.g., CetuGEX™+2 pg), the QIAamp DNA Investigator Kit yielded consistently lower values than did the comparison kit. If there had been a negative value, then the NucleoSpin Tissue XS Kit would have had a lower overall Cp value. It was decided to continue working with the QIAamp DNA Investigator Kit.
3. Improvement of the DNA elution in the QIAamp DNA Investigator Kit
   Because the QIAamp DNA Investigation Kit turned out to be the most suitable one for the clean-up of CetuGEX™, it was used for DNA preparation henceforth. Because the Cp value of CetuGEX™ after clean-up was in part still very high, it was necessary to increase the DNA content. To this end, experiments were conducted to improve the elution of DNA in the QIAamp DNA Investigator Kit. All experiments were conducted with 4 ml CetuGEX™. A Proteinase K digestion and an ultrafiltration were performed prior to the clean-up. A clean-up using the QIAamp DNA Investigator Kit was performed afterwards. The only factor that was changed in the individual experiments was the elution. The evaluation was performed by means of human qPCR.
   It was hypothesized that the DNA might dissolve better at 45° C. than at room temperature (RT) and thus that more DNA would be eluted. The effect of the buffer was therefore tested at 45° for two samples. The two comparison samples were incubated at RT, as recommended in the manual.
   It was hypothesized that there would be an elution improvement with a double elution. With three samples, after the last centrifugation the eluate was applied to the column again, incubated for 5 min, and then centrifuged again for elution.
   Test for improvement of elution by means of prolonging the incubation time of the elution buffer. Another parameter that could have improved the elution was the reaction time of the elution buffer. Hence the elution buffer was incubated at room temperature for 5 min, 10 min, and 15 min for three samples in each case. Because of very high Cp values, which led to the assumption that the CetuGEX sample used did not contain any DNA, another experiment under controlled DNA addition was conducted. In this experiment, 4 ml CetuGEX, to which 2 pg standard DNA were added, were used as samples in each case.
   An experimental series was conducted with the QIAamp DNA Investigator Kit in the next step, with the aim of improving the DNA elution of the silica membrane. With these experiments, an attempt was made to maximize DNA yields. The temperature during the incubation of the elution buffer was varied. The incubations took place either at room temperature or at 45° C. As can be seen in Table 3, the Cp values were lowered significantly by the increased temperature.

TABLE 3
Cp values regarding the improvement of the elution result with the QIAamp
Investigator Kit by means of temperature. The elution was performed twice at
room temperature and twice at 45° C. CetuGEX ™ was used as the sample;
Ø: Mean; σ Cp: Standard Deviation; red: outlier that was not considered.

| | Room Temperature | Room Temperature | 45° C. | 45° C. |
|---|---|---|---|---|
| | Sample 1 | Sample 2 | Sample 1 | Sample 2 |
| Cp 1 | 33.75 | 32.70 | 31.03 | 31.51 |
| Cp 2 | 35.43 | 31.97 | 31.57 | 30.48 |
| Cp 3 | 35.77 | 32.67 | 31.76 | 31.62 |
| Ø Cp | 34.98 | 32.45 | 31.45 | 31.57 |
| σ Cp | 1.08 | 0.41 | 0.38 | 0.08 |

It seemed that applying the eluate to the column again after eluting the sample (double elution) would be very promising in terms of yield improvement. In theory, additional DNA should dissolve from the column. However, the Cp values obtained by using this method increased or were comparable (see Table 4) and thus indicated no or even an adverse effect.

TABLE 4
Cp values of the elution improvement of the QIAamp Investigator Kit by means of double
elution. Single elution: Elution one time according to the manual. With double elution, the
eluate was applied to the column again, and also incubated and spun down as instructed in
the manual. CetuGEX ™ was used as the sample; Ø: Mean; σ Cp: Standard Deviation.

| | Single Elution | Single Elution | Double Elution | Double Elution |
|---|---|---|---|---|
| | Sample 1 | Sample 2 | Sample 1 | Sample 2 |
| Cp 1 | 34.56 | 33.55 | 34.49 | 40.00 |
| Cp 2 | 34.20 | 33.42 | 34.97 | 34.69 |
| Cp 3 | 34.83 | 33.61 | 34.76 | 33.82 |
| Ø Cp | 34.53 | 33.53 | 34.74 | 36.17 |
| σ Cp | 0.32 | 0.10 | 0.24 | 3.35 |

Increasing the reaction time of the elution buffer was considered as a third possible way to improve the elution. Hence for this purpose, samples were also incubated for 10 and 15 minutes in addition to the 5 min recommended in the protocol. The results of this experiment are shown in Table 5. No trend was discernible on the basis of mean Cp values.

TABLE 5
Cp values from the experiments on the improvement of the elution of the QIAamp
Investigator Kit by means of longer reaction time. The elution buffer was incubated
on the column for 5 min, 10 min, and 15 min at room temperature. CetuGEX ™
was used as the sample; Ø, σ Cp and red: see the legends of the preceding tables.

| | 5 Minutes | 5 Minutes | 10 Minutes | 10 Minutes | 15 Minutes | 15 Minutes |
|---|---|---|---|---|---|---|
| | Sample 1 | Sample 2 | Sample 1 | Sample 2 | Sample 1 | Sample 2 |
| Cp 1 | 37.01 | 36.72 | 37.25 | 37.41 | 35.86 | 37.93 |
| Cp 2 | 36.50 | 34.88 | 36.57 | 36.71 | 34.87 | 36.27 |
| Cp 3 | 35.80 | 34.97 | 36.40 | 36.81 | 34.05 | 36.12 |
| Ø Cp | 36.44 | 34.93 | 36.74 | 36.98 | 34.93 | 36.20 |
| σ Cp | 0.61 | 0.06 | 0.45 | 0.38 | 0.91 | 0.11 |

Because the Cp values for the experiment on the improvement of the elution conditions by prolonging the incubation times of the elution buffer were higher than 35.8 several times, the absence of DNA in this CetuGEX sample could not be ruled out. In order to carry out a DNA detection reliably, the preceding experiment was therefore modified by spiking the CetuGEX material with 2 pg human DNA (concentration of the spike: 2 pg DNA per 4 ml). With regard to elution performance, these results (compare Table 6) were likewise comparable to those without addition of DNA (see Table 6).

TABLE 6
Cp values from the experiments on the improvement of the elution of the QIAamp
Investigator Kit by means of longer reaction time. The elution buffer was incubated on the
column for 5 min, 10 min, and 15 min at room temperature in each case. CetuGEX ™ + 2pg
human DNA was used as the sample; Ø, σ Cp, and red: see the legends of the preceding tables.

| | | 5 Minutes | 10 Minutes | 10 Minutes | 15 Minutes | 15 Minutes |
|---|---|---|---|---|---|---|
| | 5 Minutes | CetuGEX | CetuGEX | CetuGEX | CetuGEX | CetuGEX |
| | CetuGEX | +2 pg | +2 pg | +2 pg | +2 pg | +2 pg |
| | +2 pg human | human | human | human | human | human |
| | DNA | DNA | DNA | DNA | DNA | DNA |
| Cp 1 | 33.49 | 35.54 | 34.86 | 35.97 | 33.16 | 36.14 |
| Cp 2 | 33.32 | 34.04 | 34.76 | 34.64 | 32.94 | 35.72 |
| Cp 3 | 33.07 | 34.64 | 34.47 | 34.20 | 32.84 | 35.05 |
| Ø Cp | 33.29 | 34.74 | 34.70 | 34.94 | 32.98 | 35.64 |
| σ Cp | 0.21 | 0.75 | 0.20 | 0.92 | 0.16 | 0.55 |

Incidentally, the concentration factor is ca. 32-fold when using this kit.

### Example 2

1. Measurement of Residual DNA of FSH Samples Using Human qPCR
   FSH-GEX™ samples were measured undiluted, as described in Example 1.1. In addition, an inhibition control was performed in order to discern possible inhibitions of the PCR. A human standard DNA concentration of 90 fg/μL was used as the control spike. The measurements were performed in triplicate with the same sample on two different days in order to ensure the quality of the results.
   The residual DNA of FSH-GEX™ was quantified using human qPCR for the first time. When measuring a sample type for the first time, it must be tested whether it is necessary to prepare the sample for the measurement, and if so in what way. For testing, the FSH-GEX™ samples were measured undiluted. The results of this can be seen in Table 7.

TABLE 7
Results of the quantification of an undiluted FSH-GEX ™ sample using
human qPCR. The measurements were performed on two different days;
Ø = Mean; σ = Standard Deviation. See text for the calculation of Ø Conc.

| | | | | DNA | Ø DNA | Ø |
|---|---|---|---|---|---|---|
| | | Ø | σ | Content | Content | Conc. |
| | Cp | Cp | Cp | (fg) | (fg) | (fg/μL) |
| Measurement 1 | 33.25 | 33.49 | 0.26 | 14.40 | 9.96 | 3.32 |
| | 33.77 | | | 5.17 | | |
| | 33.44 | | | 10.30 | | |
| Measurement 2 | 36.19 | 36.87 | 0.61 | 2.88 | 1.49 | 0.50 |
| | 37.06 | | | 0.97 | | |
| | 37.37 | | | 0.63 | | |

### Example Calculation of the Mean Concentration (Ø Conc.) in the Measurement of a Sample:

Mean content=9.96 fg
Sample volume per well=3 μl
Dilution factor of the sample: 1

$${∅}_{onc .} = \frac{∅ Content}{Volume} \times Dilution Factor = \frac{9.96 fg}{3 µl} \times 1 = 3.32 \frac{fg}{µl}$$

An incubation control was also performed in order to discern possible inhibitions (see Table 8 for the results).

TABLE 8
Results of the inhibition control of the quantification of an
undiluted FSH sample using human qPCR. The measurements were
performed on two different days; Ø = Mean; σ = Standard Deviation;
see text for the calculation of Ø Conc. Spike.

| | | | | | Ø | Ø |
|---|---|---|---|---|---|---|
| | | | | DNA | Total | Conc. |
| | | Ø | σ | Content | DNA | Spike |
| | Cp | Cp | Cp | (fg) | Content (fg) | (fg/μL) |
| Measurement | 30.85 | 30.71 | 0.24 | 94.20 | 107.00 | 97.04 |
| 1 | 30.84 | | | 94.80 | | |
| | 30.43 | | | 132.00 | | |
| Measurement | 31.99 | 31.95 | 0.08 | 78.90 | 81.30 | 79.81 |
| 2 | 32.00 | | | 78.60 | | |
| | 31.85 | | | 86.40 | | |

Example Calculation of the Mean Concentration of the Spike (Ø Conc. Spike) with Inhibition Control:
Volume of the spike=1 μl
Total DNA content=107.00 fg
Content, DNA of the sample (not of the inhibition control)=9.96 fg

$$∅ Conc . Spike = \frac{Total DNA Content - Sample DNA Content}{Spike Volume} = \frac{107.00 fg - 9.96 fg}{1 µl} = 97.04 \frac{fg}{µl}$$

The inhibition control was used to calculate the recovery of the spike. A PCR was deemed successful if the recovery was 80-120%. Table 9 shows that the recovery rates were within this range.

TABLE 9
Spike recovery rates, human qPCR of FSH-GEX ™
samples. See text for calculations.

| | Ø Conc. Spike | Target Spike | Recovery | Target Recovery |
|---|---|---|---|---|
| | (fg/μL) | (fg/μl) | (%) | (%) |
| Sample 1 | 97.04 | 90.00 | 108. | 80-120 |
| Sample 2 | 79.81 | 90.00 | 89 | 80-120 |

### Example Calculation of the Recovery Rate of the Spike of an Inhibition Control:

Ø Conc. Spike=97.04 fg/μl
Target Spike=90.00 fg/μl

$$Recovery = \frac{∅ Conc . Spike}{Target Spike} \times 100 \% = \frac{97.04 \frac{fg}{µl}}{90.00 \frac{fg}{µl}} \times 100 \% = 108 \%$$

### Example 3

### Generation of the Standard Curve and Reproducibility

Table 10 shows the Cp values obtained in generating the standard curve (three-fold determination) for the respective DNA concentrations of the E1-DNA of the 5 different measurements.

TABLE 10
Reproducibility of the E1-DNA as a standard. Shown are the Cp values of the 5
measurements (three-fold determination in each case) of the E1-DNA of the 27.5 ng/μL to 2.75
fg/μL concentrations und of the NTC under optimized qPCR conditions; outliers, which were not
included in the calculation of the mean, are in red; outliers were defined as such because they
deviated excessively from both the expected Cp value and the ones obtained from the other
measurements; the mean (Ø) of all Cp values at the E1-DNA concentration given in column 1 is
shown in the next-to-last column; the standard deviation (STDEV) calculated from the obtained
Cp values is given in the last column.

| Concentration | Cp Values | Cp Values | Cp Values | Cp Values | Cp Values | Ø Cp | |
|---|---|---|---|---|---|---|---|
| E1-DNA | Measurement 1 | Measurement 2 | Measurement 3 | Measurement 4 | Measurement 5 | Values | STDEV |
| 27.5 ng/μL | 10.60 | 10.50 | 10.52 | 10.58 | 10.51 | 10.58 | 0.05 |
| | 10.61 | 10.63 | 10.59 | 10.59 | 10.55 | | |
| | 10.62 | 10.56 | 10.52 | 10.71 | 10.57 | | |
| 2.75 ng/μL | 13.93 | 13.91 | 13.96 | 13.92 | 13.93 | 13.92 | 0.04 |
| | 13.95 | 13.91 | 13.95 | 13.89 | 13.86 | | |
| | 13.91 | 13.93 | 14.02 | 13.92 | 13.88 | | |
| 275 pg/μL | 17.55 | 17.51 | 17.49 | 17.52 | 17.48 | 17.50 | 0.03 |
| | 17.50 | 17.54 | 17.52 | 17.49 | 17.49 | | |
| | 17.54 | 17.48 | 17.49 | 17.50 | 17.46 | | |
| 27.5 pg/μL | 21.03 | 21.00 | 21.04 | 20.97 | 21.02 | 21.02 | 0.04 |
| | 21.09 | 21.08 | 21.04 | 20.96 | 21.02 | | |
| | 21.07 | 21.00 | 21.03 | 21.01 | 20.99 | | |
| 2.75 pg/μL | 24.67 | 24.56 | 24.63 | 24.54 | 24.58 | 24.59 | 0.04 |
| | 24.64 | 24.62 | 24.55 | 24.53 | 24.58 | | |
| | 24.54 | 24.54 | 24.64 | 24.58 | 24.61 | | |
| 275 fg/μL | 28.05 | 28.06 | 28.12 | 28.13 | 28.51 | 28.27 | 0.28 |
| | 28.23 | 28.12 | 28.06 | 28.47 | 28.52 | | |
| | 27.95 | 28.13 | 28.18 | 28.43 | 29.02 | | |
| 27.5 fg/μL | 31.65 | 31.86 | 31.64 | 31.50 | 31.64 | 31.76 | 0.31 |
| | 31.99 | 31.64 | 31.81 | 31.79 | 31.82 | | |
| | 31.55 | 32.42 | 32.22 | 31.11 | 31.81 | | |
| 2.75 fg/μL | 35.00 | 35.05 | 35.8 | 33.82 | 34.84 | 35.27 | 0.69 |
| | 35.00 | 36.15 | 40 | 35.03 | 35.90 | | |
| | 35.00 | 35.41 | 30.45 | 34.68 | 35.55 | | |
| NTC | / | / | 40 | 36.06 | / | 38.04 | 2.16 |
| | 35 | 38.1 | 40 | 36.04 | / | | |
| | / | / | / | / | / | | |

In the 27.5 ng/μL to 2.75 pg/μL range, the reproducibility of the Cp values within the three-fold determination and also between the individual measurements is good. Table 11 shows that the Cp values of the 275 fg/μL to 2.75 fg/μL concentrations are still reproducible with acceptable variations.

TABLE 11
Summary of the reproducibility of the E1-DNA, including
standard deviation. Shown are the Cp means, including the
standard deviation resulting from the measurement values

| Concentration | log DNA | Ø Cp | Standard |
|---|---|---|---|
| E1-DNA | Concentration | values | Deviation |
| 27500000 fg/μL | 7.44 | 10.58 | 0.05 |
| 2750000 fg/μL | 6.44 | 13.92 | 0.04 |
| 275000 fg/μL | 5.44 | 17.50 | 0.03 |
| 27500 fg/μL | 4.44 | 21.02 | 0.04 |
| 2750 fg/μL | 3.44 | 24.59 | 0.04 |
| 275 fg/μL | 2.44 | 28.27 | 0.28 |
| 27.5 fg/μL | 1.44 | 31.76 | 0.31 |
| 2.75fg/μL | 0.44 | 35.27 | 0.69 |
| NTC | | 38.04 | 2.16 |

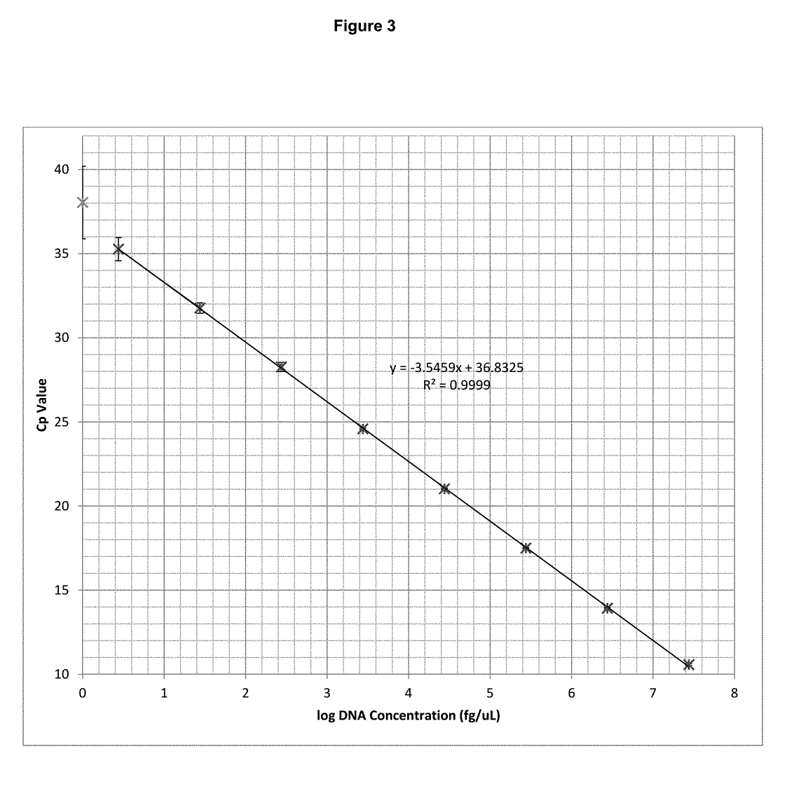

FIG. 3 shows the standard line (generated by Excel) for Table 11. The log of the respective E1-DNA concentration is plotted against the respective mean, including the standard deviation.
The coefficient of determination R²≥0.99 illustrates the linear correlation of the E1-DNA to the Cp values. The efficiency of the standard curve is calculated using the following formula:

$$E = {10}^{\frac{- 1}{Slope}}$$

The slope of the standard line calculated by Excel is −3.5459. Accordingly, the efficiency is:

$$E = {10}^{\frac{- 1}{- 3.5459}}$$

$$E = 1.914$$

With an efficiency of 1.914, the standard line lies within the range (≥1.8) deemed acceptable by Roche. Hence the E1-DNA is suitable as a standard and for generating a standard line. However, it is important to define the range in which the standard deviation of a given concentration may lie before an unequivocal quantifiability can be assumed. Lastly, a standard curve was generated on the LightCycler480 by measuring 6 replicates per concentration (27.5 ng/μL to 2.75 fg/μL). The Cp values obtained, including standard deviations, are given in Table 12.

TABLE 12
Values for generating the standard curve with E1-DNA on the
LightCycler480. Shown are the Cp values measured for the respective
concentration, and the resulting mean and standard deviation. Values in red
are outliers (Grubbs' outlier test). They were not used for calculating the
means and standard deviations, nor for generating the standard line.

| Concentration | | | Ø Cp | Standard |
|---|---|---|---|---|
| E1-DNA | Cp Values | | Values | Deviation |
| 27.5 ng/μL | 10.30 | 10.30 | 10.30 | 0.02 |
| | 10.33 | 10.25 | | |
| | 10.31 | 10.29 | | |
| 2.75 ng/μL | 13.70 | 13.72 | 13.75 | 0.03 |
| | 13.76 | 13.80 | | |
| | 13.74 | 13.75 | | |
| 275 pg/μL | 17.23 | 17.22 | 17.24 | 0.03 |
| | 17.20 | 17.22 | | |
| | 17.28 | 17.26 | | |
| 27.5 pg/μL | 20.80 | 20.82 | 20.82 | 0.03 |
| | 20.77 | 20.88 | | |
| | 20.80 | 20.83 | | |
| 2.75 pg/μL | 24.34 | 24.43 | 24.39 | 0.06 |
| | 24.49 | 24.41 | | |
| | 24.32 | 24.33 | | |
| 275 fg/μL | 27.74 | 28.07 | 28.03 | 0.02 |
| | 28.02 | 28.04 | | |
| | 28.03 | 28.00 | | |
| 27.5 fg/μL | 31.85 | 32.03 | 31.73 | 0.30 |
| | 31.95 | 31.80 | | |
| | 31.61 | 31.12 | | |
| 2.75 fg/μL | 36.17 | 33.18 | 34.75 | 0.89 |
| | 35.00 | 34.56 | | |
| | 34.50 | 35.10 | | |
| NTC | 35.54 | Unknown | | |
| | Unknown | 37.19 | | |
| | 40 | 36.96 | | |

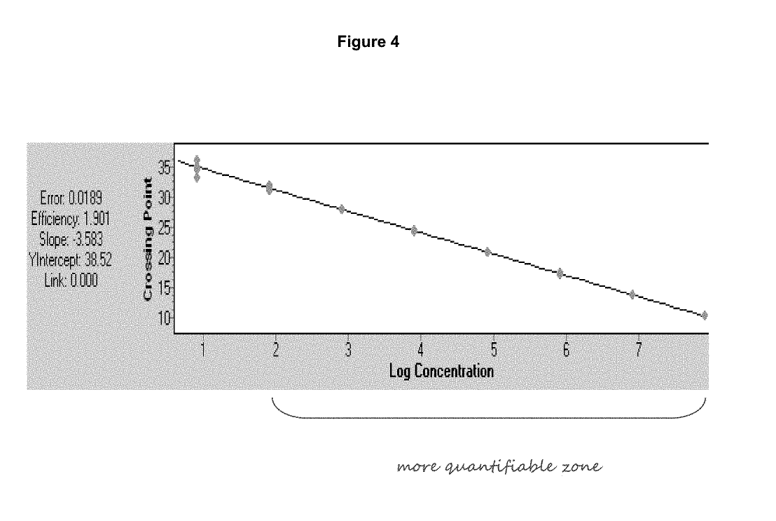

FIG. 4 shows the standard curve calculated and stored by the LigthCycler480 software. With an acceptable standard deviation of 0.45 for the quantification, the quantifiability is in the range of 27.5 ng/μL to 27.5 fg/μL and the detectability is down to 2.75 fg/μL.
An example of a commercially available kit for the detection and quantification of human DNA is the Investigator Quantiplex Kit from Qiagen (Detection limit˜1 pg/μL, quantification limit 4.9 pg/μL), in which a 146 bp fragment of an autosomal multi-copy region of the human genome is amplified. Also available is the Plexor HY System from Promega, in which a quantification limit of 3.2 pg/μL is specified.
Examples of other known assays for the detection and quantification of host cell DNA include the PicoGreen Assay, in which double-stranded DNA is detected in a non-sequence specific manner by fluorescence. The detection limit of this assay is ˜1 pg/μL.
There are not any publications or commercial kits in which a detection limit as low as the one established here is introduced. Even the quantification limit of the qPCR established herein (27.5 fg/μL) is still lower by more than 33×than the quantification limits of the methods in commercially available kits or other known methods. Even without the concentration effect of the DNA preparation, it was possible to surpass these detection limits. This step established herein lowers the detection limit of the DNA contained in the initial samples considerably further.
A further increase of the sensitivity (ca. 32-fold) is achieved with the concentration step. In the combined assay (protease splitting, concentration and enrichment of the nucleic acid), a detection limit of at least 0.086 fg/μL and a quantification limit of 0.86 fg/μL would thus be reached for the initial concentration of nucleic acid (in particular DNA) in a sample.

### Example 4

The purpose of this experiment is to confirm that the method according to the invention produces the desired results. The following experimental approaches were implemented:
Complete Procedure (CP)
Procedure without the protease step (w/o PK)
Procedure without the concentration step (w/o UF)
Procedure without enrichment of the nucleic acid (w/o SM)
   For each experimental approach, the product was spiked with two different quantities of genomic K562-DNA (final spike concentrations: 0.4 pg/mL and 40 pg/mL). The aim is to show that a reliable signal for 0.4 pg/mL is only achievable with the complete procedure.

### Materials

15 mL Falcon tubes

### Parafilm

Amicon ultrafiltration units
Microtiter plates (96-well)
Sealing film for 96-well plates

### Equipment

Water bath at 56° C.
Rotina centrifuge (Hettich)

### LightCycler 480 (Roche)

### Reagents

CHO K1 V2-Standard DNA (2 pg/uL)
K-652 DNA V2-Standard (900 pg/uL)
Proteinase K with 923 U/mL

### 10% SDS

PCR materials

### SYBR mix

Yb8F (10 uM), Yb8R (10 uM) primers
CHO1F (100 uM); CHO1R (100 uM) primers

### PCR-grade H₂O

### QIAgen DNS Investigator kit (Tag2)

AW1 buffer (in aliquots)
AW2 buffer (in aliquots)
Elution buffer

### Preparation of a K-562-DNA Dilution Series

A K-562 DNA serial dilution is performed as follows: 100 pg/uL, 10 pg/uL, 1 pg/uL, 250 fg/uL, 100 fg/uL, 50 fg/uL, 25 fg/uL and 10 fg/uL

### Spiking

Spiking is carried out according to Table 13
3.2 uL of genomic CHO DNA (V5 dilution) are added
Different quantities of the K562 standard are added
A control with a CHO DNA spike is performed.

TABLE 13
DNA Spiking Overview

| | Complete Procedure | Complete Procedure | W/O PK - | W/O PK - |
|---|---|---|---|---|
| | (CP) - 0.4 pg/mL | (CP) - 40 pg/mL | 0.4 pg/mL | 40 pg/mL |
| Sample volume | 4 mL | 4 mL | 4 mL | 4 mL |
| DNA standard | 250 fg/uL | 10 pg/uL | 250 fg/uL | 10 pg/uL |
| conc. | | | | |
| DNA standard | 6.4 uL | 16 uL | 6.4 uL | 16 uL |
| addition | | | | |
| CHO Spike | 3.2 uL | 3.2 uL | 3.2 uL | 3.2 uL |
| DNA V5 | (->0.8 pg/mL) | (->0.8 pg/mL) | (->0.8 pg/mL) | (->0.8 pg/mL) |
| (1 pg/uL) | | | | |
| addition | | | | |
| Final DNA conc. | 0.4 pg/mL | 40 pg/mL | 0.4 pg/mL | 40 pg/mL |
| | (K562) + 0.8 pg/mL | (K562) + 0.8 pg/mL | (K562) + 0.8 pg/mL | (K562) + 0.8 pg/mL |
| | (Spike) | (Spike) | (Spike) | (Spike) |

### Proteinase K Digestion

Proteinase K digestion is performed according to protocols known to persons skilled in the art.

### Ultrafiltration

Ultrafiltration is performed according to protocols known to persons skilled in the art.

### Nucleic Acid Clean-Up

DNA clean-up is performed using silica adsorption, e.g., with the QIAamp DNA Invest. Kit according to the Clean Up protocol

### Nucleic Acid Detection

| Pre-Inc.: | 1 cycle | Analysis Mode: none | | | |
|---|---|---|---|---|---|
| Amplification: | 45 cycles | Analysis Mode: Quantification | | | |
| Melting Curve: | 1 cycle | Analysis Mode: Melting Curves | | | |
| Cooling: | 1 cycle | Analysis Mode: None | | | |
| | | | | | Acqu. |
| | Target ° C. | Acqu. Mode | Hold | Ramp R. | per ° C. |
| Pre-Inc.: | 95° C. | none | 10 min. | 4.4° C./s | — |
| Amplification: | 95° C. | none | 5 s | 4.4° C./s | — |
| | 71° C. | single | 20 s | 2.2° C./s | — |
| Melting Curve: | 95° C. | none | 5 s | 4.4° C./s | — |
| | 65° C. | none | 1 min. | 2.2° C./s | — |
| | 97° C. | continuous | — | — | 7 acqu./° C. |
| Cooling: | 40° C. | none | 10 s | 1.5° C./s | — |

### Results and Evaluation

TABLE 14
qPCR Measurement and DNA Determination

| | | | | Determined | | |
|---|---|---|---|---|---|---|
| | | qPCR | Conc. | DNA | | |
| Sample | | Measurement | Factor | Concentration | | Recovery |
| no. | Designation | [pg/mL] | [-] | [pg/mL] | Target | [%] |
| 001 | CM +40 pg/mL KP | 1720 | 62.5 | 27.5 | 40.0 | 68.8 |
| 002 | CM +40 pg/mL W/O PK | NA | 25 | NA | 40.0 | 0.0 |
| 003 | CM +40 pg/mL W/O UF | 222 | 6.25 | 35.5 | 40.0 | 88.8 |
| 004 | CM +40 pg/mL W/O SM | NA | 10 | NA | 40.0 | 0.0 |
| 005 | CM +0.4 pg/mL KP | 25.4 | 62.5 | ~0.4 | 0.4 | ~100% |
| 006 | CM +0.4 pg/mL w/o PK | NA | 25 | NA | 0.4 | 0.0 |
| 007 | CM +0.4 pg/mL w/o UF | NA | 6.25 | NA | 0.4 | 0.0 |
| 008 | CM +0.4 pg/mL w/o SM | NA | 10 | NA | 0.4 | 0.0 |

NA - not available because there are no data (no signal)

Two of the four procedures tested, namely the complete procedure as well as the procedure without the use of the ultrafiltration units (Amicon Ultra 4 mL, Merck Millipore), enable a DNA measurement by means of qPCR. For a K562-DNA spike concentration of 40 pg/mL, a 69% recovery with a 62.5-fold concentration is achieved using the complete procedure. Without ultrafiltration, the recovery is around 89%, whereas the concentration factor is only 6.25-fold and thus ten times lower. The consequence of omitting the Proteinase K step or the clean-up by means of the silica membrane (e.g., QIAamp DNA Investigator Kit) is that no measurement takes place. A measurement is only possible with the complete procedure in the case of a K562-DNA concentration of 0.4 pg/ml in a CetuGEX antibody preparation. Wth the procedure and a 62.5-fold concentration, a qPCR measurement at 25.4 pg/mL (averaged Cp value: 31.84) becomes possible, which corresponds to a ca. 100% recovery.

TABLE 15
Inhibition level of the sample matrix after clean-up.
Zero inhibition control (ZIC):

| | | qPCR | | | Inhibition |
|---|---|---|---|---|---|
| IC for | | Measurement of | Target | Recovery | (y/n) |
| Sample No. | Designation of the IC | the IC [pg/ml] | [pg/ml] | [%] | over 30% |
| 001 | IC CM + 40 pg/ml KP | 2080 | 2011 | 103 | n |
| 002 | IC CM + 40 pg/ml W/O PK | NA | 291 | NA | y |
| 003 | IC CM + 40 pg/ml W/O UF | 613 | 513 | 119 | n |
| 004 | IC CM + 40 pg/ml W/O SM | NA | 291 | NA | y |
| 005 | IC CM + 0.4 pg/ml CP | 408 | 316 | 129 | n |
| 006 | IK CM + 0.4 pg/ml w/o PK | NA | 291 | NA | j |
| 007 | IK CM + 0.4 pg/ml w/o UF | 354 | 291 | 121 | n |
| 008 | IK CM + 0.4 pg/ml w/o SM | NA | 291 | NA | j |

291 pg/ml; target value corresponds to the sum of ZIC and the respective sample measurement; IC—inhibition control; NA—not available; no data available; y—yes; n—no

The inhibition levels of the samples were tested using a PCR-based K562 DNA spike (inhibition control; ˜333 pg/ml). Wth the complete procedure and the procedure without ultrafiltration, the recovery of the inhibition control is between 70 and 130%, clearly indicating that there is no inhibition. Omitting the Proteinase K step or the clean-up by means of the silica membrane leaves inhibitory substances in the cleaned sample.

1. A method for detecting a nucleic acid in a sample containing biological material that is intended to be administered to human beings, comprising
(a) Treatment of the sample with protease;
(b) Concentration of the sample;
(c) Enrichment of the nucleic acid from the sample by affinity chromatography by silica adsorption; and
(d) Detection of the nucleic acid by means of quantitative PCR.

2. The method according to claim 1, wherein the biological material originates from mammal cells, bacteria cells, or fungus cells.

3. The method according to claim 1, wherein the biological material can contain nucleic acids, proteins, carbohydrates, and/or lipids.

4. The method according to claim 1, wherein the nucleic acids can be DNA or RNA.

5. The method according to claim 1, wherein the nucleic acid can originate from a host cell that produces the biological material.

6. The method according to claim 1, wherein the biological material contains antibodies or a therapeutic protein.

7. The method according to claim 6, wherein the antibody is an antibody against EGFR, Her2, TA-MUC1, TF, or LeY.

8. The method according to claim 6, wherein the protein is FSH, hCG, hLH, hGH, Factor VII, Factor FVIIa, Factor FVIII, Factor VIIIa, Factor IX, Factor IXa, Factor X, or Factor Xa.

9. The method according to claim 1, wherein the mammal cells are human cells, primate cells, mouse cells, rat cells, hamster cells, or rabbit cells.

10. The method according to claim 9, wherein the mammal cells are PER.C6, HEK cells, NS0 cells, Vero cells, CHO cells, for example DUXB11, DG44, or CHOK1, mouse hybridoma cells, rat hybridoma cells, or rabbit hybridoma cells

11. The method according to claim 1, wherein the sample is liquid biological material or biological material dissolved or suspended in liquid.

12. The method according to claim 1, wherein the protease in step (a) is Proteinase K.

13. The method according to claim 1, wherein the concentration of the sample in step (b) is effected by means of filtration, with volume reduction.

14. The method according to claim 13, wherein the filtration is an ultrafiltration.

15. The method according to claim 1, wherein the silica adsorption is effected by means of a silica-based membrane.

16. The method according to claim 1, wherein detection of the nucleic acid by means of quantitative PCR in step (d) focuses on repetitive elements.

17. The method according to claim 16, wherein the repetitive elements are Alu sequences or Alu-equivalent sequences.

18. The method according to claim 1, wherein in step (d), use is made of the primer pairs with SEQ ID Nos. 1 and 2, SEQ ID No. 3 and SEQ ID No. 4, SEQ ID Nos. 5 and 6, SEQ ID Nos. 1 and 7, SEQ ID Nos.1 and 8, SEQ ID Nos.1 and 9, SEQ ID Nos.1 and 10, SEQ ID Nos. 11 and 12, or of a mixture of the primer pairs with SEQ ID Nos. 1 and 7-10.

19. The method according to claim 1, wherein the sample is a biopharmaceutical or biotechnological product.

20. The method according to claim 1, wherein the detection of a nucleic acid in the sample is quantitative.

21. The method according to claim 1, wherein the method is for detecting a nucleic acid of a host cell in biological material that is administered to human beings.

22. The method according to claim 1, wherein the method is for determining whether biological material for administration to human beings is essentially free of host cell nucleic acids, in particular DNA.

23. The method according to claim 1, wherein the method is for quantifying host cell nucleic acids in a sample.

24. A kit for carrying out a method according to claim 1, comprising
(a) Protease;
(b) Means for concentrating liquid samples;
(c) Means for the affinity chromatography based on silica adsorption, and
(d) DNA polymerase and a primer pair for amplifying repetitive elements in DNA.

25. A primer pair with SEQ ID Nos. 1 and 2, SEQ ID Nos. 1 and 7, SEQ ID Nos.1 and 8, SEQ ID Nos.1 and 9, SEQ ID Nos.1 and 10, SEQ ID Nos. 11 and 12, or a mixture of the primer pairs with SEQ ID Nos. 1 and 7-10.


SEQUENCE LISTING (12 sequences)

SEQ ID NO: 1 - DNA, 22 bp, artificial
  Alu-Forward primer
aggagatcgagaccatcctggc

SEQ ID NO: 2 - DNA, 21 bp, Artificial
  Alu-Reverse primer
tggcgcaatctcggctcactg

SEQ ID NO: 3 - DNA, 22 bp, artificial
  Alu-Forward primer
cgaggcgggtggatcatgaggt

SEQ ID NO: 4 - DNA, 20 bp, artificial
  Alu-Reverse primer
tctgtcgcccaggccggact

SEQ ID NO: 5 - DNA, 20 bp, artificial
  Alu equivalent Forward primer
tggagagatggctcgaggtt

SEQ ID NO: 6 - DNA, 21 bp, artificial
  Alu equivalent Reverse primer
tggttgctgggaattgaactc

SEQ ID NO: 7 - DNA, 21 bp, artificial
  Alu-Reverse primer
tggtgcaatctcggctcactg

SEQ ID NO: 8 - DNA, 21 bp, artificial
  Alu-Reverse primer
tggcgcgatctcggctcactg

SEQ ID NO: 9 - DNA, 21 bp, artificial
  Alu-Reverse primer
tggcgctatctcggctcactg

SEQ ID NO: 10 - DNA, 21 bp, artificial
  Alu-Reverse primer
tggcgcaatctcagctcactg

SEQ ID NO: 11 - DNA, 22 bp, artificial
  Alu-Forward Primer
  misc (2)..(2): r is g or a
  misc (3)..(3): r is g or a
  misc (4)..(4): h is a or c
  misc (5)..(5): r is g or a
  misc (6)..(6): d is a, t or g
  misc (7)..(7): k is t or g
  misc_feature (8)..(8): n is a, c, g, or t
  misc (9)..(9): r is g or a
  misc (10)..(10): w is a or t
  misc (13)..(13): s is c or g
  misc (16)..(16): k is t or g
  misc (18)..(18): y is c or t
arrhrdknrwgascakcytggc

SEQ ID NO: 12 - DNA, 21 bp, artificial
  Alu-Reverse Primer
  misc (1)..(1): y is t or c
  misc (3)..(3): b is g, t or c
  misc (4)..(4): y is c or t
  misc (5)..(5): r is g or a
  misc (6)..(6): b is c, t or g
  misc (7)..(7): d is a, g or t
  misc (10)..(10): y is c or t
  misc (12)..(12): b is c, t or g
  misc_feature (13)..(13): n is a, c, g, or t
  misc (14)..(14): b is c, t or g
  misc (19)..(19): y is c or t
  misc (20)..(20): y is c or t
ygbyrbdatytbnbctcayyg